(19) United States
(12) Patent Application Publication
(10) Pub. No.: US 2021/0153757 A1
(43) Pub. Date: May 27, 2021

(54) ELECTRONIC DEVICE FOR PERFORMING COMMUNICATION WITH WEARABLE DEVICE FOR RECEIVING BIOMETRIC INFORMATION

(71) Applicant: Samsung Electronics Co., Ltd., Gyeonggi-do (KR)

(72) Inventors: Taehan JEON, Gyeonggi-do (KR); Hongji LEE, Gyeonggi-do (KR); Hyejung SEO, Gyeonggi-do (KR)

(21) Appl. No.: 17/045,652

(22) PCT Filed: Mar. 8, 2019
(86) PCT No.: PCT/KR2019/002691

§ 371 Date: Oct. 6, 2020

(30) Foreign Application Priority Data
Apr. 26, 2018 (KR) 10-2018-0048303

(51) Int. Cl. A61B 5/024; G06F 21/32; A61B 5/0245; A61B 5/00

(52) U.S. Cl. CPC A61B 5/02416; A61B 5/681; A61B 5/0245; G06F 21/32

(57) ABSTRACT

The present disclosure provides an electronic device including a sensor which measures first biometric information, a communication unit which receives second biometric information measured by at least one wearable device, and a processor operatively connected with the sensor and the communication unit, and the processor calculates a correlation between the first biometric information and the second biometric information, grant, to at least one wearable device, an authenticated authority based on the correlation, the authenticated authority being an authority to approve performance of a specified operation, and sets an authenticated authority level that is a step classified based on reliability of the authenticated authority. In addition to the above, various embodiments identified using the specification are possible.

### TECHNICAL FIELD

Embodiments of the present disclosure relate to a technology for receiving and analyzing biometric information from a wearable device that receives biometric information.

### BACKGROUND ART

Electronic devices have evolved into portable devices as the requirement for portability is demanded. In recent years, among electronic devices, wearable devices have been developed that is capable of being worn on the body. The wearable device is worn on a certain body part of a user. For example, a watch-type device is worn on the wrist of the user, an earbud-type device is worn on the ears of the user, and a glass-type device is worn on the eyes of the user. The user may use the wearable device at the desired time while wearing it on the body. Therefore, the wearable device supports an easier control environment for the user.

The wearable device receives biometric signals of the user. The wearable device measures biometric signals such as photoplethysmogram (PPG) and electrocardiogram (ECG). A method for measuring a biometric signal using a wearable device is a noninvasive method.

The biometric signal measured using the wearable device includes information related to health of the user. Therefore, the wearable device supports mobile healthcare to the user by using the biometric signal. In addition, the user may purchase goods or use financial services using the wearable device. After completing authentication of the wearable device, the user may receive health-related information included in the biometric signal, or may purchase goods or use financial services.

### DISCLOSURE OF THE INVENTION

### Technical Problem

The electronic device may determine whether or not the wearable device is worn using the biometric signal measured by the wearable device. However, the electronic device may not determine whether or not the user of the electronic device and the wearer of the wearable device are the same person. The user performs authentication by directly inputting a password, pattern, and PIN number specified in the wearable device. In addition, an authenticated authority level of the wearable device may not be set without a separate input to the wearable device. Accordingly, it may not be convenient for the user to perform authentication of the wearable device.

### Technical Solution

According to an aspect of the present disclosure, there is provided an electronic device including a sensor which measures first biometric information, a communication unit which receives second biometric information measured by at least one wearable device, and a processor operatively connected with the sensor and the communication unit, and the processor calculates a correlation between the first biometric information and the second biometric information, grant, to at least one wearable device, an authenticated authority based on the correlation, the authenticated authority being an authority to approve performance of a specified operation, and sets an authenticated authority level that is a step classified based on reliability of the authenticated authority.

According to another aspect of the present disclosure, there is provided a method for controlling an electronic device, including: pairing with at least one wearable device; measuring first biometric information by the electronic device; requesting the at least one wearable device to measure biometric information; measuring second biometric information by the at least one wearable device; receiving the second biometric information from the at least one wearable device and comparing the first biometric information with the second biometric information; and calculating a correlation between the first biometric information and the second biometric information, granting, to at least one wearable device, an authenticated authority that is an authority to approve performance of a specified operation, based on the correlation, and setting an authenticated authority level that is a step classified based on reliability of the authenticated authority.

According to another aspect of the present disclosure, there is provided a method for controlling an electronic device, including: connecting with a first wearable device; receiving first biometric information measured by the first wearable device; connecting with a second wearable electronic device; and receiving second biometric information measured by the second wearable electronic device, calculating a correlation between the first biometric information and the second biometric information, granting, to the second wearable device, an authenticated authority that is an authority to approve performance of a specified operation, based on the correlation, and setting an authenticated authority level that is a step classified based on reliability of the authenticated authority.

### Advantageous Effects

With the embodiments disclosed in the present disclosure, it is possible to determine whether the user of the electronic device and the wearer of the wearable device are the same person or another person who are different from the user of the electronic device is wearing the wearable device by using the biometric signal measured by the wearable device. If the user of the electronic device and the wearer of the wearable device are the same person, the electronic device may grant, to the wearable device, the first authenticated authority level that is a high authenticated authority level including a security function or a payment function. Therefore, the electronic device may set the authenticated authority level of the wearable device without a separate input to the wearable device.

Besides, various effects may be provided that are directly or indirectly identified using the present disclosure.

### BRIEF DESCRIPTION OF THE DRAWINGS

FIG. 1 is a diagram illustrating an electronic device and a first wearable device according to an embodiment.

FIG. 2 is a flowchart illustrating the operation of the electronic device according to an embodiment.

FIG. 3 is a flowchart illustrating an operation of the electronic device according to another embodiment.

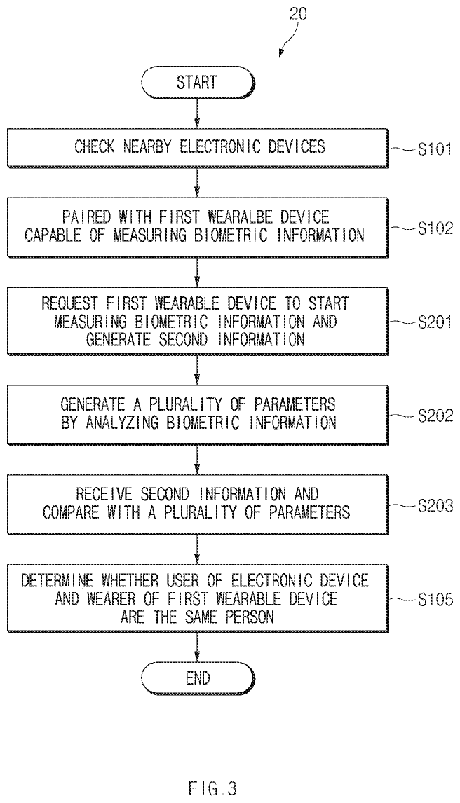

FIG. 4 is a diagram illustrating first biometric information and second biometric information according to an embodiment.

FIG. 5 is a flowchart illustrating a process of analyzing biometric information by the electronic device according to an embodiment.

FIG. 6 is a diagram illustrating a PPG signal according to an embodiment.

FIG. 7 is a flowchart illustrating a process of analyzing biometric information by the electronic device according to another embodiment.

FIG. 8 is a block diagram illustrating an analysis unit according to an embodiment.

FIG. 9 is a flowchart illustrating a process of granting reliability between the electronic device and the first wearable device according to an embodiment.

FIG. 10 is a diagram illustrating heart rates per minute for each of a plurality of sections of the electronic device and the first wearable device according to an embodiment.

FIG. 11 is a flowchart illustrating a process of authenticating the first wearable device by the electronic device according to an embodiment.

FIG. 12 is a flowchart illustrating a process of authenticating the first wearable device by the electronic device according to another embodiment.

FIG. 13 is a flowchart illustrating a process of authenticating the first wearable device by a second wearable device according to an embodiment.

FIG. 14 is a flowchart illustrating a process of authenticating the second wearable device by the first wearable device according to an embodiment.

FIG. 15 is a block diagram illustrating an electronic device in a network environment according to various embodiments.

With respect to the description of the drawings, the same or similar reference signs may be used for the same or similar elements.

### MODE FOR CARRYING OUT THE INVENTION

Hereinafter, various embodiments disclosed in the present disclosure will be described with reference to the accompanying drawings. However, this is not intended to limit the present disclosure to the specific embodiments, and it is to be construed to include various modifications, equivalents, and/or alternatives of embodiments of the present disclosure.

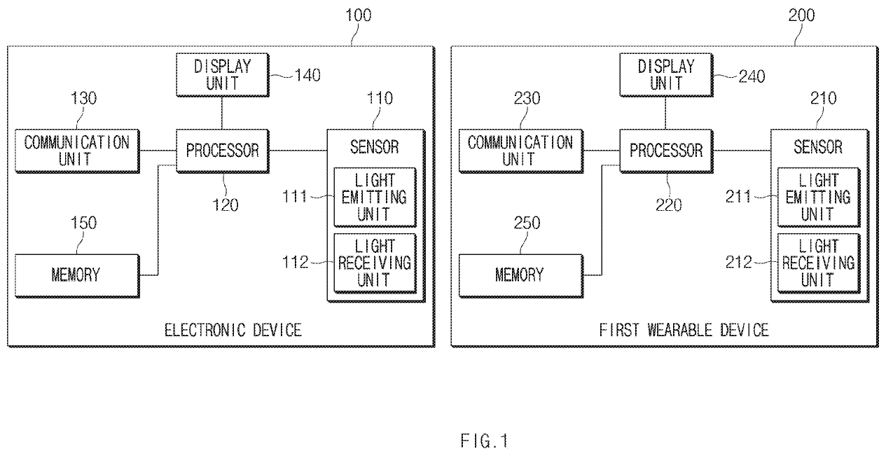

FIG. 1 is a diagram illustrating an electronic device 100 and a first wearable device 200 according to an embodiment.

The electronic device 100 according to an embodiment may include a sensor 110, a processor 120, a communication unit 130, a display unit 140, and a memory 150. The electronic device 100 according to an embodiment may be a portable terminal such as a smartphone or a tablet. However, the electronic device 100 is not limited thereto, and may be a patch type wearable device, a sticker type device, or an implantable device.

In an embodiment, the sensor 110 may be disposed on the surface of the electronic device 100. The sensor 110 may be disposed on a surface adjacent to the user. The sensor 110 may measure biometric information of the user (e.g., heart rate information, photoplethysmogram (PPG) information, or electrocardiogram (ECG) information). The sensor 110 may be a PPG sensor or an ECG sensor.

If the sensor 110 according to an embodiment is the PPG sensor, the sensor 110 may measure a change rate of blood flowing through a blood vessel due to the heartbeat of the user. The sensor 110 may measure a light absorption rate of the blood vessel that changes due to blood flow that changes by a heartbeat of a heart user or a light absorption rate of a skin adjacent to the blood vessel. The sensor 110 may include at least one light emitting unit 111 and at least one light receiving unit 112.

In an embodiment, at least one light emitting unit 111 may include a plurality of light emitting diodes (LEDs). The at least one light emitting unit 111 may include one or more LEDs of an infrared (IR) LED, a red LED, a green LED, and a blue LED. For example, the at least one light emitting unit 111 may be formed of a plurality of infrared LEDs and a plurality of red LEDs. In this case, at least some of the plurality of infrared LEDs and the plurality of red LEDs constituting the at least one light emitting unit 111 may output light toward the skin of the user.

In an embodiment, the at least one light receiving unit 112 may receive the light reflected from at least one of a blood vessel of the user and skin of the user, in the light output from the at least one light emitting unit 111. The at least one light receiving unit 112 may convert the received light into an electrical signal. If the intensity of light received by the at least one light receiving unit 112 changes, the intensity and waveform of the electrical signal may change. At least one light receiving unit 112 may be a photo diode.

In an embodiment, the sensor 110 may generate at least one piece of biometric information by using the electrical signal converted from the light received using the at least one light receiving unit 112. The at least one biometric information may be a PPG signal waveform or an ECG signal waveform measured using the sensor 110. Alternatively, the at least one piece of biometric information may be numerical values such as a heart rate (HR), a blood flow rate, and blood pressure generated by analyzing a signal measured using the sensor 110.

In an embodiment, the sensor 110 may convert the measured biometric information into digital data. The sensor 110 may transmit, to the processor 120, the biometric information that has been converted into digital data.

In an embodiment, the processor 120 may be operatively connected with the sensor 110, the communication unit 130, the display unit 140, and the memory 150. The processor 120 may receive at least one piece of biometric information measured by the sensor 110. The processor 120 may emit at least one piece of biometric information using the communication unit 130. The processor 120 may display at least one piece of biometric information using the display unit 140. The processor 120 may include an application processor (AP). Alternatively, the processor 120 may include a sensor hub that operates at lower power than the AP.

In an embodiment, the processor 120 may acquire, from the sensor 110, biometric information that has been converted into digital data. The processor 120 may acquire biometric information according to the instruction content stored in the memory 150. The processor 120 may store the acquired biometric information in the memory 150. The processor 120 may analyze biometric information stored in the memory 150. The processor 120 may analyze the biometric information according to the instruction details stored in the memory 150.

In an embodiment, the processor 120 may calculate a plurality of feature points (e.g., a peak amplitude of the biometric signal, a peak period of the biometric signal, and an interval between the starting point and the peak point of the biometric signal) by using the biometric information converted into digital data. The processor 120 may analyze the biometric information waveform by extracting feature points of the acquired biometric information waveform. The processor 120 may check a user of the electronic device 100 using the result of analyzing the feature points. The processor 120 may compare one or more pieces of biometric information with each other using the feature points. The processor 120 may transmit the biometric information to the communication unit 130.

In an embodiment, the processor 120 may convert biometric information stored in the memory 150 into an analog biometric signal and may transmit it to the display unit 140 to display a waveform. The processor 120 may determine whether the user carries the electronic device 100 in his or her hand. For example, if the processor 120 performs an operation of detecting a touch on the display of the display unit 140, it may determine that the user is holding the electronic device 100 in his or her hand normally. Alternatively, if an event occurs by which the sensor 110 determines that the skin of the user is in proximity, the processor 120 may determine that the user is holding the electronic device 100 in his or her hand normally. If the user is holding the electronic device 100 in his or her hand normally, the processor 120 may receive biometric information and convert it into a biometric signal. If the user is holding the electronic device 100 in his or her hand normally, the processor 120 may transmit the biometric signal to the display unit 140.

In an embodiment, the communication unit 130 may receive at least one piece of biometric information from the processor 120. The processor 130 may emit the biometric information. The communication unit 130 may be functionally connected with at least one wearable device. The electronic device 100 may be wirelessly connected with the first wearable device 200 using the communication unit 130. For example, the communication unit 130 may support communication between the electronic device 100 and the first wearable device 200. The communication unit 130 may transmit the biometric information to the first wearable device 200 by a communication method such as Bluetooth, BLE, ANT+, Wi-Fi, Cellular (LTE, 5G, LTE-M1, NB-IoT), and NFC.

In an embodiment, the display unit 140 may receive at least one piece of biometric information from the processor 120 and visually display it. The display unit 140 may display at least one piece of biometric information in a graph in a time domain or a graph in a frequency domain. Alternatively, the display unit 140 may display at least one piece of biometric information three-dimensionally by synthesizing it with a three-dimensional human body structure. The display unit 140 may be implemented as a liquid crystal display (LCD), an organic light emitting display (OLED), a quantum dot LED, a mini LED, a micro LED, or the like.

In an embodiment, the memory 150 may receive, from the processor 120, biometric information that has been converted into digital data. The memory 150 may store biometric information that has been converted into digital data. The memory 150 may transmit previously stored biometric information to the processor 120. The memory 150 may store instructions such that the processor 120 acquires and analyzes biometric information.

The first wearable device 200 according to an embodiment may include a sensor 210, a processor 220, a communication unit 230, a display unit 240, and a memory 250. The first wearable device 200 according to an embodiment may be a smartwatch, earbuds, a glass, a belt, a shoe, a ring, or the like. The configuration and functions of the sensor 210, the processor 220, the communication unit 230, the display unit 240, and the memory 250 of the first wearable device 200 are substantially the same as the configuration and function of the sensor 110, the processor 120, the communication unit 130, the display unit 140, and the memory 150 of the electronic device 100, and thus a detailed description thereof will be omitted.

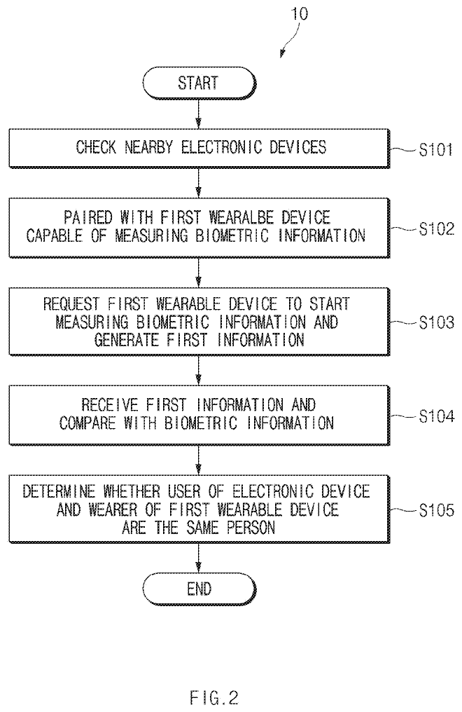

FIG. 2 is a flowchart 10 illustrating an operation of the electronic device 100 according to an embodiment.

In operation S101, the electronic device 100 according to an example may check nearby electronic devices. The electronic device 100 may discover nearby electronic devices by using the communication unit 130. For example, the electronic device 100 may receive connection signals and model information from nearby electronic devices. The electronic device 100 may emit a request signal to request nearby electronic devices to transmit connection signals and model information. The nearby electronic device may be a wearable device. However, the present disclosure is not limited thereto, and the nearby electronic device may be any electronic device located in a short distance from the electronic device 100.

In operation S102, the electronic device 100 according to an example may be paired with the first wearable device 200 capable of measuring biometric information. The electronic device 100 may check whether the discovered electronic device has the capability to measure biometric information. The electronic device 100 may establish a communication connection with the first wearable device 200 by using the communication unit 130.

In an embodiment, the first wearable device 200 may be worn on the body of a wearer. The first wearable device 200 may measure biometric information of the wearer by using the sensor 210. The first wearable device 200 may be paired with the electronic device 100 by using the communication unit 230.

In operation S103, the electronic device 100 according to an example may start measuring biometric information. If an analysis unit 122 determines that the user is carrying the electronic device 100, the electronic device 100 may measure biometric information by using the sensor 110. For example, the biometric information may be a PPG signal waveform or an ECG signal waveform measured using the electronic device 100.

In operation S103, the electronic device 100 according to an example may request the first wearable device 200 to measure biometric information. The first wearable device 200 may receive the request from the electronic device 100 and may generate first information. The first information may be biometric information measured by the first wearable device 200. For example, the first information may be a PPG signal waveform or an ECG signal waveform measured using the first wearable device 200.

In operation S104, the electronic device 100 according to an example may receive the first information from the first wearable device 200. The communication unit 230 of the first wearable device 200 may output a signal including the first information. The communication unit 130 of the electronic device 100 may receive the first information and transmit it to the processor 120. The processor 120 of the electronic device 100 may compare the first information measured by the first wearable device 200 with the biometric information measured by the electronic device 100.

In operation S105, the electronic device 100 according to an example may determine whether the user of the electronic device 100 and the wearer of the first wearable device 200 are the same person. The processor 120 of the electronic device 100 may determine a degree of matching between the biometric information and the first information.

For example, if the sensor 110 of the electronic device 100 and the sensor 210 of the first wearable device 200 are PPG sensors, the processor 120 of the electronic device 100 may compare the PPG signal measured by the electronic device 100 with the PPG signal measured by the first wearable device 200. The processor 120 of the electronic device 100 may analyze at least one of a correlation between and phases of the two PPG signals. The processor 120 of the electronic device 100 may determine whether the two PPG signals are signals measured from the same person by using at least one of the correlation between and phases of the two PPG signals.

In an embodiment, the electronic device 100 may grant an authenticated authority to the first wearable device 200 based on the correlation between the electronic device 100 and the first wearable device 200. The authenticated authority may be an authority to approve the performance of a specified operation by the electronic device 100 or the first wearable device 200. For example, the authenticated authority is the authority to approve access to personal information of the user stored in the electronic device 100 or the first wearable device 200, or an authority to approve payment using the electronic device 100 or the first wearable device 200.

In an embodiment, a first authenticated authority may be directly granted by the user to the electronic device 100. The user may grant the authenticated authority to the electronic device 100 by performing an authentication procedure. For example, the authenticated authority may be granted to the electronic device 100 in a way in which the user enters designated information (e.g., input of a password, pattern, and PIN number) such that the electronic device 100 checks the user.

In an embodiment, the electronic device 100 may set an authenticated authority level of the first wearable device 200, which is a step classified based on the reliability of the authenticated authority. The reliability of the authenticated authority may be calculated based on the correlation between the electronic device 100 and the first wearable device 200. The authenticated authority level may be classified into a plurality of levels based on the reliability of the authenticated authority. For example, the authenticated authority level granted to the first wearable device 200 may be classified into a first authenticated authority level having the same reliability as that of the electronic device 100 for which user authentication is completed by performing an authentication procedure and a second authentication authority level having a lower reliability or more limited reliability than the first authenticated authority level. The authenticated authority level granted to the first wearable device 200 may set a range of operations that the first wearable device 200 is capable of performing.

FIG. 2 is a flowchart 20 illustrating an operation of the electronic device 100 according to another embodiment. Operation S101, operation S102, and operation S105 of the electronic device 100 according to another embodiment are substantially the same as operations S101, S102, and S105 of the electronic device 100 according to an example, and thus detailed description thereof will be omitted.

In operation S201, the electronic device 100 according to an example may request the first wearable device 200 to start measuring biometric information and generate second information. The second information may be numerical information generated using first information, which is biometric information measured by the first wearable device 200. For example, the second information may include numerical values such as a heart rate (HR), a blood flow rate, or blood pressure generated by analyzing a biometric signal (e.g., a PPG signal waveform or an ECG signal waveform) measured using the first wearable device 200.

In operation S202, the electronic device 100 according to an example may generate a plurality of parameters by analyzing the biometric information. The plurality of parameters may be numerical values such as the heart rate, the blood flow rate, or the blood pressure generated by analyzing the biometric signal (e.g., the PPG signal waveform or the ECG signal waveform) measured using the electronic device 100.

In operation S203, the electronic device 100 according to an example may receive the second information from the first wearable device 200. The communication unit 230 of the first wearable device 200 may output a signal including the second information. The communication unit 130 of the electronic device 100 may receive the second information and transmit it to the processor 120. The processor 120 of the electronic device 100 may compare a plurality of parameters generated by the electronic device 100 with the second information received from the first wearable device 200.

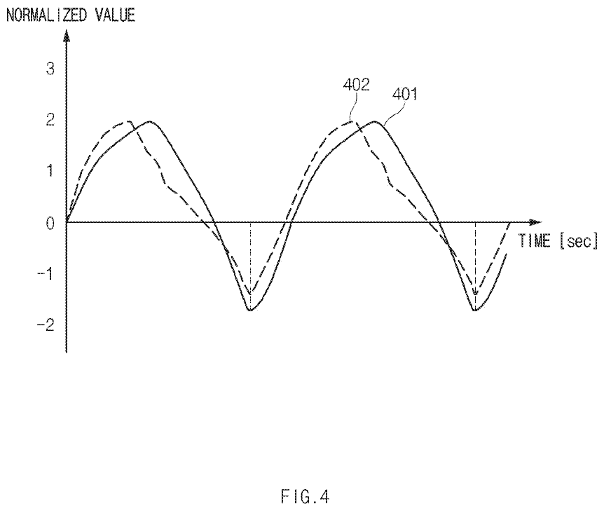

FIG. 4 is a diagram illustrating first biometric information 401 and second biometric information 402 according to an embodiment. The first biometric information 401 may be biometric information (e.g., biometric information of FIG. 2) measured using the electronic device 100. The second biometric information 402 may be biometric information (e.g., first information of FIG. 2) measured using the first wearable device 200.

In an embodiment, while the electronic device 100 measures the first biometric information 401, the first wearable device 200 may measure the second biometric information 402. The first biometric information 401 and the second biometric information 402 may be measured at the same time. The electronic device 100 may perform analysis by receiving the second biometric information 402 measured simultaneously with the first biometric information 401.

In an embodiment, the electronic device 100 may normalize the first biometric information 401 and the second biometric information 402 during a preset section. The normalized first biometric information 401 and second biometric information 402 may have a preset amplitude (e.g., a normalized value of −2 or more and 2 or less). In the normalized first biometric information 401 and second biometric information 402, the highest or lowest waveform point may be adjusted to have the same highest point or lowest point.

In an embodiment, the electronic device 100 may calculate the correlation between the first biometric information 401 and the second biometric information 402 during the preset section. The correlation between the first biometric information 401 and the second biometric information 402 may be calculated by using a difference value between the first biometric information 401 and the second biometric information 402. As the difference between the first biometric information 401 and the second biometric information 402 is smaller, the correlation between the first biometric information 401 and the second biometric information 402 may be higher.

In an embodiment, the electronic device 100 may measure a change rate of each of the first biometric information 401 and the second biometric information 402. The correlation between the first biometric information 401 and the second biometric information 402 may be calculated by comparing the change rate of the first biometric information 401 with the change rate of the second biometric information 402. As the difference between the change rate of the first biometric information 401 and the change rate of the second biometric information 402 is smaller, the correlation between the first biometric information 401 and the second biometric information 402 may be higher.

In an embodiment, the correlation calculated when the user of the electronic device 100 and the wearer of the first wearable device 200 are the same person is between the user of the electronic device 100 and the first wearable device 200 may be higher than the correlation calculated when the user of the electronic device 100 and the user of the wearable device 200 are different people.

For example, if the user of the electronic device 100 and the wearer of the first wearable device 200 are the same person, a correlation of 0.8 or more and 0.95 or less may be calculated when a simple correlation versus analysis is performed. On the other hand, if the user of the electronic device 100 and the wearer of the first wearable device 200 are different people, the correlation may be calculated to be less than 0.7 when the simple correlation versus analysis is performed.

In an embodiment, the electronic device 100 may compare the calculated correlation with the correlation specified using the processor 120. If the calculated correlation is equal to or greater than the specified correlation, the electronic device 100 may determine that the user of the electronic device 100 and the wearer of the first wearable device 200 are the same person. If the calculated correlation is less than the specified correlation, the electronic device 100 may determine that the user of the electronic device 100 and the wearer of the first wearable device 200 are different people.

For example, when the specified correlation is 0.75, if the correlation of 0.8 or more and 0.95 or less is calculated when the simple correlation versus analysis is performed, the electronic device 100 may determine that the user of the electronic device 100 and the wearer of the wearable device 200 are the same person. In addition, when the specified correlation is 0.75, if the correlation of less than 0.7 is calculated when the simple correlation versus analysis is performed, the electronic device 100 may determine that the user of the electronic device 100 and the wearer of the wearable device 200 are different people.

In an embodiment, if the user of the electronic device 100 or the wearer of the first wearable device 200 has movement, the electronic device 100 may change the specified correlation. If the identification or discrimination power of the biometric signal of the user of the electronic device 100 or the wearer of the first wearable device 200 increases, the electronic device 100 may decrease the specified correlation. If the identification or discrimination power of the biometric signal of the user of the electronic device 100 or the wearer of the first wearable device 200 increases, the electronic device 100 may determine whether the user of the electronic device 100 and the wearer of the first wearable device 200 are the same person even if the correlation is lower than the originally specified correlation.

For example, if the user of the electronic device 100 or the wearer of the first wearable device 200 walks or runs, the user or the heart rate of the user may increase. Accordingly, the identification or discrimination power based on the heart rate waveform of the user of the electronic device 100 or the wearer of the first wearable device 200 may increase. In addition, the noise of the heart rate waveform due to the movement of the user of the electronic device 100 or the wearer of the first wearable device 200 may also increase. The processor 120 of the electronic device 100 may reduce the specified correlation from 0.75 to 0.65 in consideration of increased identification or discrimination power and increased noise. If the correlation of 0.65 or more is calculated when the simple correlation versus analysis is performed, the electronic device 100 may determine that the user of the electronic device 100 and the wearer of the first wearable device 200 are the same person.

In an embodiment, if the user of the electronic device 100 or the wearer of the first wearable device 200 has movement, it may be easier to determine whether the user of the electronic device 100 and the wearer of the first wearable device 200 are the same person even if the correlation specified in the electronic device 100 is lowered. If the identification or discrimination power of the biometric signal of the user of the electronic device 100 or the wearer of the first wearable device 200 increases, the electronic device 100 may perform a comparison with the change rate of the peak period of the biometric signal waveform, as well as the correlation based on the shape of the biometric signal waveform. Accordingly, even if the specified correlation is lowered, the electronic device 100 may more accurately analyze the biometric signal waveform to determine whether the user of the electronic device 100 and the wearer of the first wearable device 200 are the same person.

In an embodiment, the electronic device 100 may calculate a phase and additional parameters of each of the first biometric information 401 and the second biometric information 402 during the preset section. The electronic device 100 may compare the phase of the first biometric information 401 with the phase of the second biometric information 402. Alternatively, the electronic device 100 may compare at least one parameter of the first biometric information 401 with at least one parameter of the second biometric information 402. When the phase and additional parameters of each of the first biometric information 401 and the second biometric information 402 are calculated during the preset section, the electronic device 100 may determine whether the user of the electronic device 100 and the wearer of the first wearable device 200 are the same person more accurately than when the simple correlation versus analysis is performed.

For example, if the first biometric information 401 and the second biometric information 402 are PPG signals, the electronic device 100 may compare the first biometric information 401 with the second biometric information 402 at every preset time interval. For example, the electronic device 100 may compare two PPG signals at intervals of 100 ms. If the PPG signals are compared at a time interval shorter than the preset specified time, continuous analysis results for the PPG signals may be obtained. Accordingly, the electronic device 100 may determine in real time whether the user is wearing the first wearable device 200 on the body.

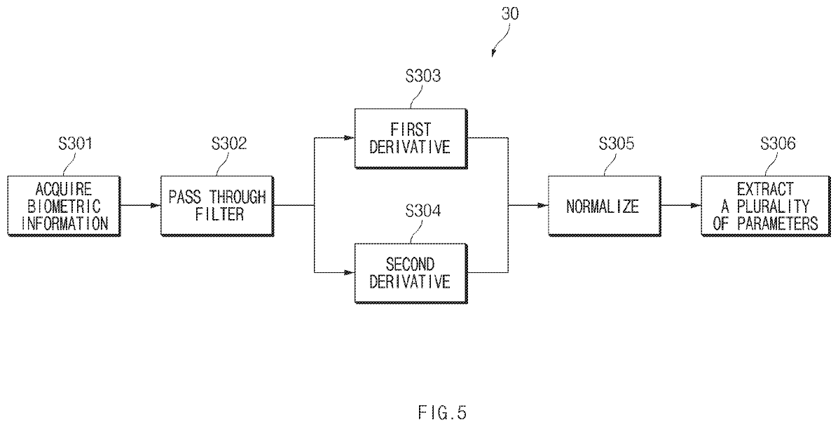

FIG. 5 is a flowchart 30 illustrating a process of analyzing biometric information 401 by the electronic device 100 according to an embodiment.

In operation S301, the electronic device 100 according to an example may acquire the biometric information 401. The sensor 110 of the electronic device 100 may measure the biometric information 401 of the user. The processor 120 of the electronic device 100 may receive the measured biometric information 401 from the sensor 110. The biometric information 401 may be analyzed in a time domain.

In operation S302, the electronic device 100 according to an example may pass the biometric information 401 through a filter. The biometric information 401 may pass through a low-pass filter in order to remove noise. The biometric information 401 passing through the low-pass filter may consist of only a preset biometric signal among biometric signals of the user. For example, the biometric information 401 passing through the low-pass filter may consist of only the PPG signal.

In operation S303, the electronic device 100 according to an example may apply a first derivative to the biometric information 401. The electronic device 100 may extract features of the biometric information 401 by using the first derivative. The electronic device 100 may extract one or more of the amplitude of the waveform of the biometric information 401, the time interval of the waveform of the biometric information 401, or the change rate of the waveform of the biometric information 401 by applying the first derivative to the biometric information 401.

In operation S304, the electronic device 100 according to an example may apply a second derivative to the biometric information 401. The electronic device 100 may extract features of the biometric information 401 by using the second derivative. The electronic device 100 may extract a change rate of the slope of the waveform of the biometric information 401 by applying the second derivative to the biometric information 401. The electronic device 100 may perform the second derivative of the biometric information 401 separately from the first derivative of the biometric information 401.

In operation S305, the electronic device 100 according to an example may normalize the biometric information 401. The electronic device 100 may convert a peak value and an average value of the biometric information 401 into preset normal values. For example, the electronic device 100 may convert the peak value of the biometric information 401 to 1 and the average value to 0.

In operation S306, the electronic device 100 according to an example may extract a plurality of parameters from the biometric information 401. The electronic device 100 may extract parameters included in the amplitude of the waveform of the biometric information 401, parameters included in the interval of the waveform, and parameters included in the area of the waveform.

In an embodiment, if the biometric information 401 is the PPG signal, the amplitude of the waveform of the biometric information 401 may include parameters such as a systolic peak, a diastolic peak, a dicrotic notch, a ratio between peaks, and a ratio of peak differences. The interval of the waveform of the biometric information 401 may include parameters such as a pulse interval, an interval between systolic peaks, a systolic peak time, a dicrotic notch time, a diastolic peak time, and a time between systolic and diastolic peaks. The area of the biometric information 401 may include parameters such as a blood flow rate and an area ratio of a systolic section and a diastolic section. The electronic device 100 may determine whether the user of the electronic device 100 and the wearer of the first wearable device 200 are the same person by comparing the extracted parameters with the parameters transmitted from the first wearable device 200.

In an embodiment, the electronic device 100 may synchronize the PPG signal measured by the electronic device 100 with the PPG signal measured by the first wearable device 200. For this synchronization, the electronic device 100 may synchronize the starting point of the PPG signal by using an external stimulus such as light or a wireless signal. For example, the electronic device 100 may form a marker for synchronizing the PPG signal using a flash.

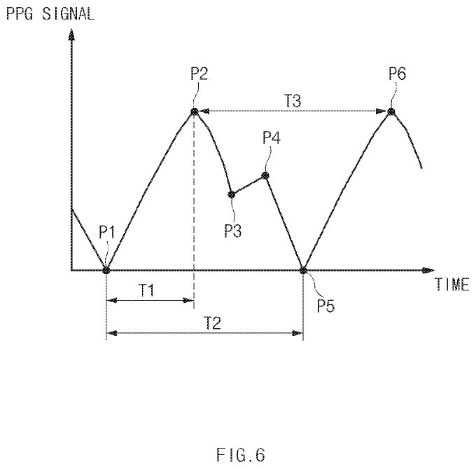

FIG. 6 is a diagram illustrating a PPG signal according to an embodiment. The PPG signal according to an example may have first to sixth points P1 to P6 and first to third sections T1 to T3 on the time axis.

In an embodiment, the PPG signal may have a minimum amplitude value at the first point P1. In the PPG signal waveform, the first point P1 may be defined as a base point.

In an embodiment, the PPG signal may have a first section T1 from the first point P1 to the second point P2. The PPG signal may increase in magnitude during the first section T1. In an embodiment, the PPG signal may have a maximum amplitude value at the second point P2. The second point P2 in the PPG signal waveform may be defined as a systolic peak. In the PPG signal waveform, the first section T1 may be defined as a systolic peak time.

In an embodiment, the amplitude of the PPG signal may decrease from the second point P2 to the third point P3. The third point P3 in the PPG signal waveform may be defined as a dicrotic notch. The amplitude of the third point P3 may be larger than the amplitude of the first point P1 and may be smaller than the amplitude of the second point P2.

In an embodiment, the amplitude of the PPG signal may increase from the second point P2 to the third point P3. The fourth point P4 in the PPG signal waveform may be defined as a diastolic peak. The amplitude of the fourth point P4 may be larger than the amplitude of the third point P3 and may be smaller than the amplitude of the second point P2.

In an embodiment, the amplitude of the PPG signal may decrease from the fourth point P4 to the fifth point P5. In the PPG signal waveform, the fifth point P5 may be defined as the base point of the next waveform. In the PPG signal waveform, a section from the first point P1 to the fifth point P5 may be defined as a second section T2. In the PPG signal waveform, the second section T2 may be defined as a pulse interval.

In an embodiment, the amplitude of the PPG signal may increase from the fifth point P5 to the sixth point P6. In the PPG signal waveform, the sixth point P6 may be defined as a systolic peak of the next waveform. In the PPG signal waveform, a section from the second point P2 to the sixth point P6 may be defined as a third section T3. In the PPG signal waveform, the third section T3 may be defined as an interval between systolic peaks.

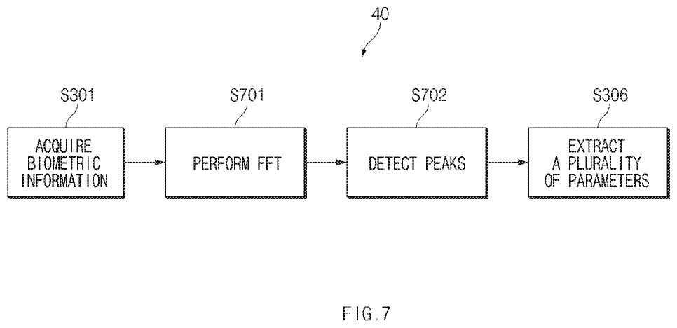

FIG. 7 is a flowchart illustrating a process of analyzing biometric information 401 by the electronic device 100 according to another embodiment. Operations S301 and S306 of a process of analyzing the biometric information 401 by the electronic device 100 according to another embodiment are substantially the same as operations S301 and S306 of the process of analyzing the biometric information 401 by the electronic device 100 according to an embodiment, and thus a detailed description thereof will be omitted.

In operation S701, the electronic device 100 according to an example may perform a fast Fourier transform (FFT). The biometric information 401 measured by the sensor 110 of the electronic device 100 may be analyzed in a frequency domain. The electronic device 100 may divide the biometric information 401 into frequency components by performing the FFT.

In operation S702, the electronic device 100 according to an example may detect a plurality of peaks. The electronic device 100 may define, as peaks, frequency components having frequency magnitudes of an integer multiple of a specified frequency which is preset from the biometric information 401 divided into frequency components.

In an embodiment, the electronic device 100 may extract a plurality of parameters from a plurality of peaks. The electronic device 100 may extract a plurality of features from frequency components. The plurality of parameters extracted by the electronic device 100 may include a peak index, a peak value, and the like.

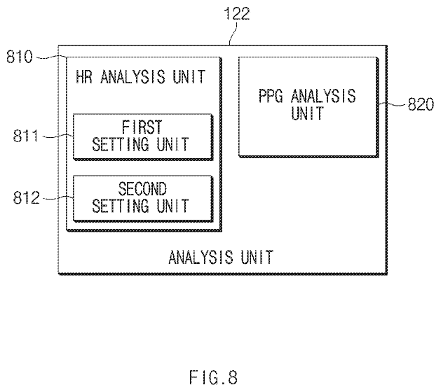

FIG. 8 is a block diagram illustrating an analysis unit 122 according to an embodiment. The analysis unit 122 according to an example may include a heart rate (HR) analysis unit 810 and a PPG analysis unit 820.

In an embodiment, the HR analysis unit 810 may measure a heart rate trend (HR trend) of the first biometric information 401 measured by the electronic device 100 and a heart rate trend of the second biometric information 402 measured by the first wearable device 200. For example, the HR analysis unit 810 may include an engine that receives a PPG signal and analyzes a change trend of a heart rate. The HR analysis unit 810 may use the correlation between the heart rate trend measured by the electronic device 100 and the heart rate trend measured by the first wearable device 200 to set the reliability between the electronic device 100 and the first wearable device 200. The HR analysis unit 810 may include a first setting unit 811 and a second setting unit 812.

In an embodiment, the first setting unit 811 may include an algorithm for changing a level according to a duration time. If the heart rate trend of the biometric information 401 and the heart rate trend of the first information 402 are changed such that they match for a duration time of a preset time or longer, the first setting unit 811 may grant high reliability to the first wearable device 200. If the heart rate trends of the user of the electronic device 100 and the wearer of the first wearable device 200 match for the preset time or longer, the electronic device 100 may grant a first authenticated authority level to the first wearable device 200. The first authenticated authority level may be an authenticated authority level having the same reliability as the electronic device 100 for which user authentication is completed. The first authenticated authority level may be the highest authenticated authority level among the authenticated authority levels that the electronic device 100 grants to the first wearable device 200. For example, the first authenticated authority level may be an authenticated authority level having the authority to perform a security function or a payment function (e.g., a payment function using a payment application such as Samsung Pay).

In an embodiment, the second setting unit 812 may include an algorithm for changing a level according to a change rate in the heart rate. When the change rate in the heart rate is high means when a user moves actively in a state of wearing the electronic device 100 and the first wearable device 200. In addition, the change rate in the heart rate when the user moves actively may be more diverse than the change rate in the heart rate when the user is normal. Accordingly, if the change rates in the heart rates are changed equally in a high state, the reliability of the fact that the electronic device 100 and the first wearable device 200 are worn by the same user may increase.

In an embodiment, if the change rate of the heart rate measured by the electronic device 100 and the change rate of the heart rate measured by the first wearable device 200 are equal to or greater than a preset specified amount of change, the second setting unit 812 may grant high reliability to the wearable device 200. If the change rates of the heart rates of the user of the electronic device 100 and the wearer of the first wearable device 200 are equal to or greater than the specified amount of change, the electronic device 100 may grant the first authenticated authority level to the first wearable device 200.

In an embodiment, the second setting unit 812 may differently designate whether to omit the request for additional authority according to the change rate of the heart rate. If the change rate of the heart rate measured by the electronic device 100 and the change rate of the heart rate measured by the first wearable device 200 are equal to or greater than a preset specified amount of change, the second setting unit 812 may grant the first authenticated authority level to the first wearable device 200 without a separate additional request for authority.

In an embodiment, the PPG analysis unit 820 may include an algorithm for analyzing the correlation of PPG trends. The PPG analysis unit 820 may use the correlation between the PPG signal measured by the electronic device 100 and the PPG signal measured by the first wearable device 200 to set the reliability between the electronic device 100 and the first wearable device 200.

In an embodiment, the analysis unit 122 may reflect both the result of setting the reliability of the HR analysis unit 810 and the result of setting the reliability of the PPG analysis unit 820 to set the reliability between the electronic device 100 and the first wearable device 200. If the PPG waveform measured by the electronic device 100 and the PPG waveform measured by the first wearable device 200 are the same for a duration of a preset time or longer, the analysis unit 122 may further analyze the heart rate trend measured by the electronic device 100 and the heart rate trend measured by the first wearable device 200. After checking whether the user of the electronic device 100 and the wearer of the first wearable device 200 are the same person by using the analysis result comparing the PPG waveforms, the analysis unit 122 may use the analysis result comparing the heart rate trends to determine whether the electronic device 100 grants high reliability to the first wearable device 200.

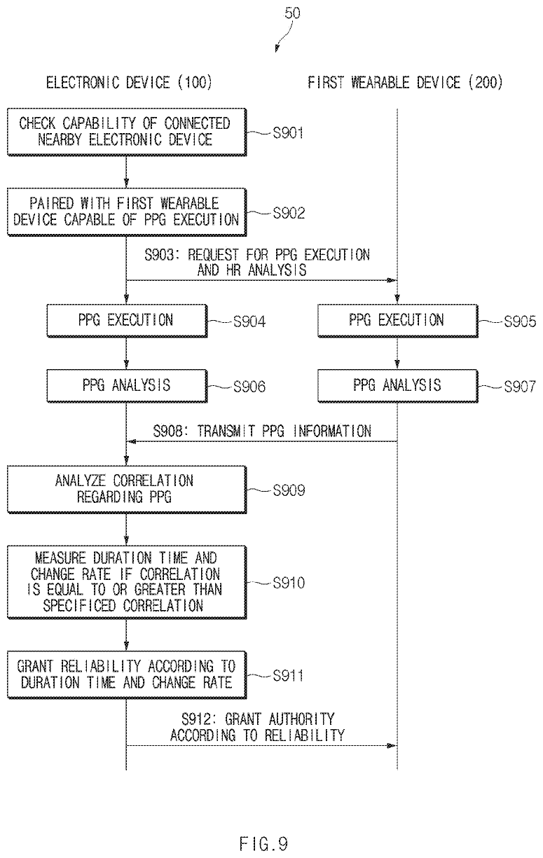

FIG. 9 is a flowchart 50 illustrating a process of granting reliability between the electronic device 100 and the first wearable device 200 according to an embodiment.

In operation S901, the electronic device 100 according to an example may check the capability of the connected nearby electronic device. The electronic device 100 may check whether the nearby electronic device is able to measure a biometric signal. For example, the electronic device 100 may check whether the nearby electronic device is able to measure a PPG signal.

In operation S902, the electronic device 100 according to an example may be paired with the first wearable device 200 capable of a PPG execution. The electronic device 100 may check whether the PPG signal is able to be measured by checking the type or model name of the first wearable device 200. The communication unit 130 of the electronic device 100 may be functionally connected with the communication unit 230 of the first wearable device 200. The electronic device 100 may be wirelessly connected with the first wearable device 200 using the communication unit 130.

In operation S903, the electronic device 100 according to an example may request the first wearable device 200 to execute the PPG and analyze the HR. The electronic device 100 may emit a wireless signal including a command for causing the first wearable device 200 to start measuring the PPG signal of the wearer of the first wearable device 200 by using the communication unit 130. The electronic device 100 may emit a wireless signal including a command for causing the first wearable device 200 to start measuring the heart rate of the wearer of the first wearable device 200 by using the communication unit 130.

In operation S904, the electronic device 100 according to an example may perform a PPG execution task. The electronic device 100 may measure the PPG signal of the user of the electronic device 100 by using the sensor 110.

In operation S905, the first wearable device 200 according to an example may perform the PPG execution task. The first wearable device 200 may measure the PPG signal of the wearer of the first wearable device 200 by using the sensor 210.

In operation S906, the electronic device 100 according to an example may perform the PPG analysis. The processor 120 of the electronic device 100 may calculate PPG information of the user of the electronic device 100 by analyzing the measured waveform of the PPG signal.

In operation S907, the first wearable device 200 according to an example may perform the PPG analysis. The processor 220 of the first wearable device 200 may calculate PPG information of the wearer of the first wearable device 200 by analyzing the measured waveform of the PPG signal.

In operation S908, the first wearable device 200 according to an example may transmit the PPG information to the electronic device 100. The first wearable device 200 may emit the PPG signal waveform extracted by the PPG analysis to the electronic device 100 by using the communication unit 230. The electronic device 100 may receive the emitted PPG signal waveform by using the communication unit 130.

In operation S909, the electronic device 100 according to an example may analyze the correlation between two PPG signals. The processor 120 of the electronic device 100 may compare the PPG signal of the wearer calculated by the electronic device 100 with the PPG signal of the wearer calculated by the first wearable device 200. The electronic device 100 may measure the correlation between the PPG signals by using a degree of matching and a change rate between the PPG signal of the wearer of the electronic device 100 and the PPG signal of the first wearable device 200.

In an embodiment, the electronic device 100 may determine whether the user of the electronic device 100 and the wearer of the first wearable device 200 match by using the degree of matching. If the waveform of the PPG signal measured by the electronic device 100 and the waveform of the PPG signal measured by the first wearable device 200 match, the electronic device 100 may determine that the user of the electronic device 100 and the wearer of the first wearable device 200 match. If the correlation between the PPG signals of the electronic device 100 and the first wearable device 200 is equal to or greater than the specified correlation, the electronic device 100 may determine that the user of the electronic device 100 and the wearer of the first wearable device 200 match.

In operation S910, if the correlation is equal to or greater than the specified correlation, the electronic device 100 according to an example may measure a time during which the PPG signals match and amounts of change of the PPG signals in the matched section. The electronic device 100 may perform additional measurement if the correlation of the PPG signals is equal to or greater than a specified value. The electronic device 100 may measure the time during which the PPG signal measured by the electronic device 100 and the PPG signal measured by the first wearable device 200 match by using the additional measurement. The electronic device 100 may measure the amount of change during the time when the PPG signal measured by the electronic device 100 and the PPG signal measured by the first wearable device 200 match by using the additional measurement.

In operation S911, the electronic device 100 according to an example may grant reliability according to a duration time and an amount of change. The electronic device 100 may grant high reliability to the first wearable device 200 if the time during which the PPG signal measured by the electronic device 100 and the PPG signal measured by the first wearable device 200 match is equal to or greater than a specified time by using the additional measurement. The electronic device 100 may grant high reliability to the first wearable device 200 if the PPG signal measured by the electronic device 100 and the PPG signal measured by the first wearable device 200 are changed beyond a specified amount of change while matching by using the additional measurement. If the duration times of the PPG signal measured by the electronic device 100 and the PPG signal measured by the first wearable device 200 using additional measurements are less than the specified time, the electronic device 100 may grant low reliability or limited reliability to the first wearable device 200.

In operation S912, the electronic device 100 according to an example may grant authority according to reliability. If high reliability is granted to the first wearable device 200, the electronic device 100 may trust with a high probability that the user of the electronic device 100 and the wearer of the first wearable device 200 are the same person. If the electronic device 100 trusts with a high probability that the user of the electronic device 100 and the wearer of the first wearable device 200 are the same person, the electronic device 100 may grant the first authenticated authority level to the first wearable device 200. If low reliability or limited reliability is granted to the first wearable device 200, the electronic device 100 may trust with a low probability that the user of the electronic device 100 and the wearer of the first wearable device 200 are the same person. If the electronic device 100 trusts with a low probability that the user of the electronic device 100 and the wearer of the first wearable device 200 are the same person, the electronic device 100 may grant the second authenticated authority level to the first wearable device 200.

In an embodiment, if the first authenticated authority level is granted to the first wearable device 200, the electronic device 100 may grant, to the first wearable device 200, the same authority as the authority the user has for the electronic device 100. For example, if the first authenticated authority level is granted to the first wearable device 200, the electronic device 100 may grant payment authority to the first wearable device 200. If the second authenticated authority level is granted to the first wearable device 200, the electronic device 100 may request the user to perform additional authentication before providing, to the first wearable device 200, authority the user has for the electronic device 100. If the second authenticated authority level is granted to the first wearable device 200, the electronic device 100 may not grant, to the first wearable device 200, at least some of the authority the user has for the electronic device 100.

In an embodiment, the electronic device 100 may perform the PPG analysis and may additionally perform the HR analysis that calculates a variability of the heart rate of the user. The electronic device 100 may grant more accurate reliability to the first wearable device 200 by combining the PPG analysis result and the HR analysis result.

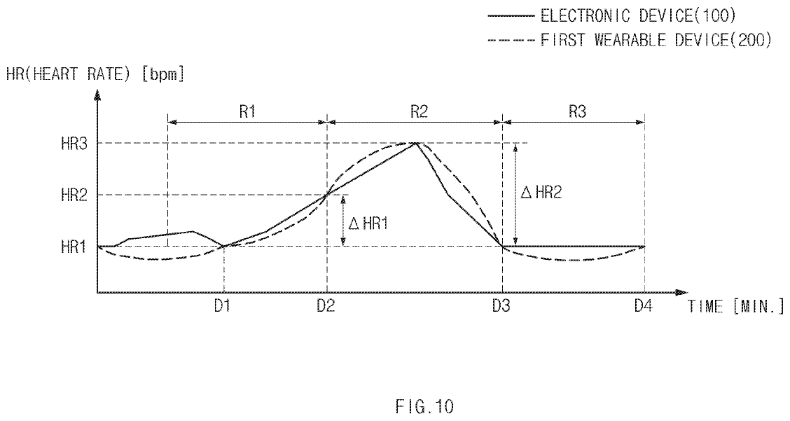

FIG. 10 is a diagram illustrating a heart rate for each of a plurality of sections of the electronic device 100 and the first wearable device 200 according to an embodiment.

In an embodiment, if the electronic device 100 uses information about a heart rate (HR), the electronic device 100 may measure and analyze a PPG signal, and then additionally generate information about the heart rate (HR) and use it. However, the present disclosure is not limited thereto, and the electronic device 100 may analyze the correlation between the electronic device 100 and each wearable device based on an electrocardiogram (ECG) signal. The electronic device 100 may display the heart rate (HR) using the display unit 140. The display unit 140 may display, in real time, a value of a beat per minute (BPM) to be measured in a plurality of sections divided by minute units.

In an embodiment, the heart rates HR of the electronic device 100 and the first wearable device 200 may change from a first heart rate HR1, which is the lowest heart rate, to a second heart rate HR2, in the first section R1, which is a section from a first time point D1 to a second time point D2. The heart rates HR of the electronic device 100 and the first wearable device 200 may change by a first amount of charge ΔHR1, which is the difference between the first heart rate HR1 and the second heart rate HR2, during the first section R1. The heart rates HR of the electronic device 100 and the first wearable device 200 may change from the second heart rate HR2 to a third heart rate HR3, which is the highest heart rate, in a second section R2, which is a section from the second time point D2 to a third time point D3, and then may change from the third heart rate HR3 to the first heart rate HR1. The heart rates HR of the electronic device 100 and the first wearable device 200 may change by a second amount of charge ΔHR2, which is the difference between the first heart rate HR1 and the third heart rate HR3, during the first section R1. The heart rates HR of the electronic device 100 and the first wearable device 200 may maintain the first heart rate HR1, which is the lowest heart rate, in a third section R3, which is a section from the third time point D3 to a fourth time point D4.

In an embodiment, the electronic device 100 and the first wearable device 200 may set a specified amount of change in the heart rate HR to be greater than the first amount of change ΔHR1 and less than the second amount of change ΔHR2. The heart rates HR of the electronic device 100 and the first wearable device 200 may change by the first amount of change ΔHR1 smaller than the specified amount of change, in the first section R1. The heart rates HR of the electronic device 100 and the first wearable device 200 may change by the second amount of change ΔHR2 greater than the specified amount of change, in the second section R2.

In an embodiment, in the first section R1, since the heart rates of the electronic device 100 and the first wearable device 200 change equally but change by the first amount of change ΔHR1 lower than the specified amount of change, the electronic device 100 may trust with a low probability that the user of the electronic device 100 and the wearer of the wearable device 200 are the same person. In the second section R2, since the heart rates of the electronic device 100 and the first wearable device 200 change equally and change by the second amount of change ΔHR2 greater than the specified amount of change, the electronic device 100 may trust with a high probability that the user of the electronic device 100 and the wearer of the wearable device 200 are the same person. In the third section R3, since the heart rates of the electronic device 100 and the first wearable device 200 are the same but stay virtually unchanged, the electronic device 100 may trust with a low probability that the user of the electronic device 100 and the wearer of the wearable device 200 are the same person.

In an embodiment, the electronic device 100 may grant low reliability or limited reliability to the first wearable device 200 in the first section R1. The electronic device 100 may grant high reliability to the first wearable device 200 in the second period R2. The electronic device 100 may grant low reliability or limited reliability to the first wearable device 200 in the third section R3.

In an embodiment, if one or more wearable devices are connected to the electronic device 100, the electronic device 100 may grant different authenticated authority level to each wearable device based on the time during which the heart rate HR stays the same as that of each wearable device and the width of change in the heart rate HR in the section where the heart rate HR is the same as that of each wearable device.

For example, the user may wear the electronic device 100 (e.g., a chest patch) and any wearable device (e.g., earbuds) simultaneously in the first section R1 and the third section R3. In addition, the user may wear the electronic device 100 and another wearable device (e.g., a smart watch) simultaneously in the second section R2 and the third section R3. In this case, in the first section R1 and the third section R3, the heart rate measured by the chest patch and the heart rate measured by the earbuds may be the same. In addition, in the second section R2 and the third section R3, the heart rate measured by the chest patch and the heart rate measured by the smart watch may be the same.

For example, the time during which the heart rate of the earbuds stays the same may be the duration time of the first section R1 or the third section R3. The time during which the heart rate of the smart watch stays the same may be a duration time of a sum of the second section R2 and the third section R3. Accordingly, the chest patch may grant the first authenticated authority level to the smart watch and may grant the second authentication authority level to the earbuds.

For another example, in the time during which the heart rate measured by the chest patch and the heart rate measured by the smart watch stay the same, the width of the change in the heart rate measured in the second section R2 may be greater than the change width of the heart rate measured in the third section R3. Accordingly, the chest patch may grant the first authenticated authority level to the smart watch in the second section R2 and may grant the second authenticated authority level to the smart watch in the third section R3.

The chest patch according to an embodiment may determine that additional authentication is not required by granting the first authenticated authority level to the smart watch in the second section R2. In the second section R2, the smart watch may perform operations with a high authenticated authority level, such as payment (e.g., payment using Samsung Pay) without additional authentication. The chest patch may determine that additional authentication is required by granting the second authenticated authority level to the smart watch in the third section R3. In the third section R3, the smart watch may additionally perform a process of requesting the user to perform an additional authentication. The smart watch may additionally perform a process of additionally measuring and analyzing other types of biometric signals (e.g., PPG signal waveform phase or blood pressure) by itself in the third section R3.

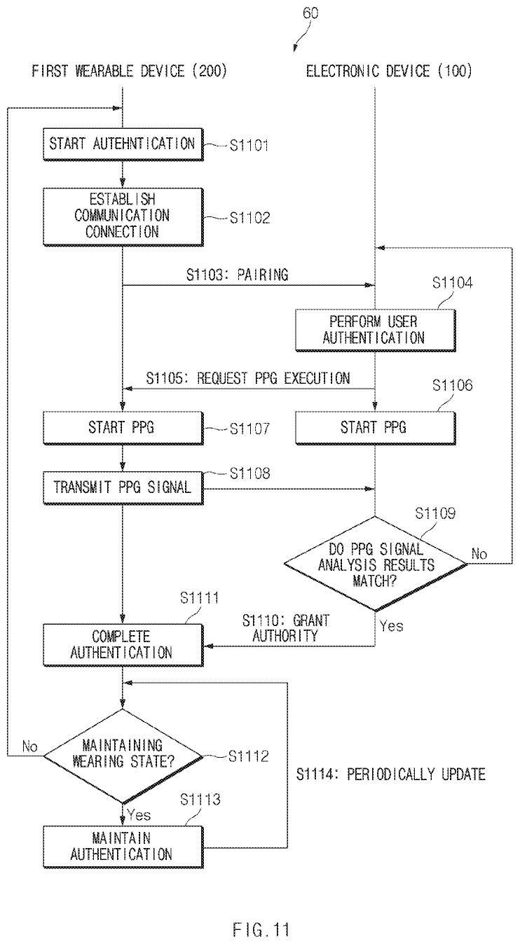

FIG. 11 is a flowchart 60 illustrating a process of authenticating the first wearable device 200 by the electronic device 100 according to an embodiment.

In operation S1101, the first wearable device 200 according to an example may start authentication. The first wearable device 200 may confirm that the user is wearing it. The first wearable device 200 may detect and sense biometric information of the user by using the sensor 210.

In operation S1102, the first wearable device 200 according to an example may establish a communication connection. The first wearable device 200 may check the electronic device 100 capable of wireless communication by using the communication unit 230. The communication unit 230 of the first wearable device 200 may prepare to perform short-range communication with the communication unit 130 of the electronic device 100.

In operation S1103, the first wearable device 200 according to an example may perform pairing with the electronic device 100. The first wearable device 200 may transmit various signals to the electronic device 100. For example, the first wearable device 200 may transmit the measured biometric information to the electronic device 100.

In operation S1104, the electronic device 100 according to an example may perform user authentication. After pairing with the first wearable device 200, the electronic device 100 may request a user who is using the electronic device 100 to perform the personal authentication. The electronic device may request the user to input information for personal authentication.

In operation S1105, the electronic device 100 according to an example may request the first wearable device 200 to execute the PPG. The electronic device 100 may emit a wireless signal including a command for causing the first wearable device 200 to start measuring the PPG signal of the wearer of the first wearable device 200 by using the communication unit 130.

In operation S1106, the electronic device 100 according to an example may perform the PPG execution task. The electronic device 100 may measure the PPG signal of the user of the electronic device 100 by using the sensor 110.

In operation S1107, the first wearable device 200 according to an example may perform the PPG execution task. The first wearable device 200 may measure the PPG signal of the wearer of the first wearable device 200 by using the sensor 210.

In operation S1108, the first wearable device 200 according to an example may transmit the PPG signal. The first wearable device 200 may emit the first information 401 including the PPG signal by using the communication unit 230. The electronic device 100 may receive the first information 402 by using the communication unit 130.

In operation S1109, the electronic device 100 according to an example may determine whether the PPG signal analysis results match. The electronic device 100 may calculate the correlation between the PPG signal measured by the electronic device 100 and the PPG signal measured by the first wearable device 200.

In an embodiment, if the calculated correlation is greater than the specified correlation, the electronic device 100 may determine that the user of the electronic device 100 and the wearer of the first wearable device 200 are the same person. If the PPG signal analysis results do not match and the calculated correlation is less than the specified correlation, the electronic device 100 may determine that the user of the electronic device 100 and the wearer of the first wearable device 200 are different people. If the PPG signal analysis results do not match, the electronic device 100 may cancel user authentication and return to a state before operation S1104 to perform user authentication.

In operation S1110, the electronic device 100 according to an example may grant authority to the first wearable device 200 if the PPG signal analysis results match. If the electronic device 100 determines that the user of the electronic device 100 and the wearer of the first wearable device 200 are the same person, the electronic device 100 may grant a preset authority to the first wearable device 200. The preset authority may be authority that the electronic device 100 has after performing user authentication.

In operation S1111, the first wearable device 200 according to an example may complete authentication. The first wearable device 200 may complete the authentication by using the authority granted by the electronic device 100.

In an embodiment, the first wearable device 200 may start an authentication procedure when a user wears it in operation S1101. The first wearable device 200 may start authentication with the same user account as the electronic device 100 in operation S1101. The first wearable device 200 may complete authentication with the same user account as the electronic device 100 in operation S1111.

In operation S1112, the first wearable device 200 according to an example may check whether the user maintains a wearing state. The first wearable device 200 may detect whether the first wearable device 200 is worn by a person every preset period. The first wearable device 200 may periodically check whether the biometric signal of the wearer is measured by using the sensor 210. If the first wearable device 200 is removed by the user and is not in a wearing state, the authentication may be revoked. If the first wearable device 200 does not maintain the wearing state, an authenticated session may be released.

In operation S1113, if the first wearable device 200 according to an example maintains the wearing state, the authentication may be maintained. After the authentication is completed, the first wearable device 200 may maintain the authenticated session while maintaining the wearing state by the user.

In operation S1114, the first wearable device 200 according to an example may periodically update the authentication maintenance. The first wearable device 200 may periodically determine whether the first wearable device 200 is worn by the user. The first wearable device 200 may determine that identity of the user of the electronic device 100 and the wearer of the first wearable device 200 is maintained by using periodic updates. The first wearable device 200 may maintain the authenticated session while the wearer is wearing the first wearable device 200 by using periodic updates.

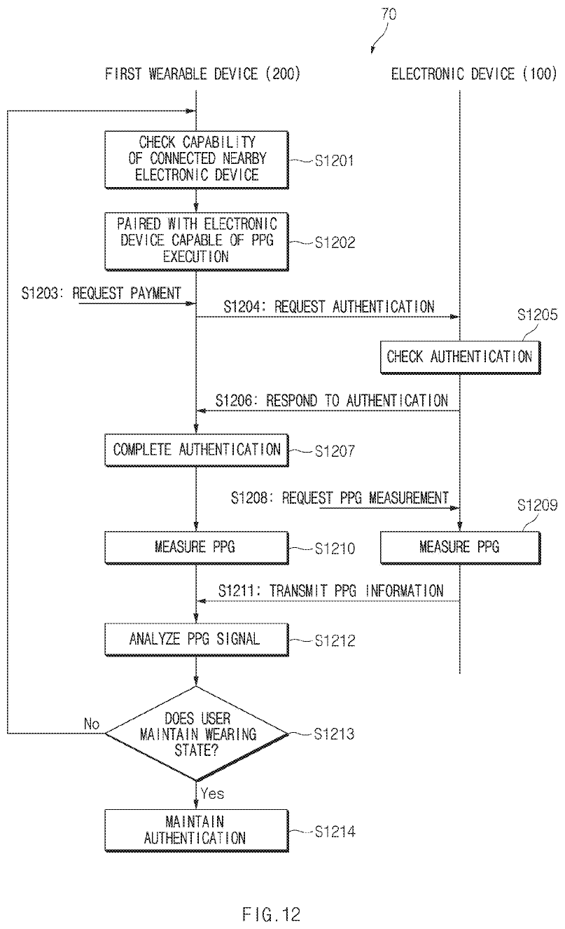

FIG. 12 is a flowchart 70 illustrating a process of authenticating the first wearable device 200 by the electronic device 100 according to another embodiment.

In operation S1201, the first wearable device 200 according to an example may check the capability of the connected nearby electronic device 100. The first wearable device 200 may establish a communication connection with the nearby electronic device 100 by using the communication unit 230. The first wearable device 200 may check the type and model name of the electronic device 100. The first wearable device 200 may determine whether the electronic device 100 is a device capable of measuring the biometric information 401.

In operation S1202, the first wearable device 200 according to an example may be paired with the electronic device 100 capable of the PPG execution. The first wearable device 200 may output a signal for requesting user authentication by using the communication unit 230.

In operation S1203, the first wearable device 200 according to an example may receive a payment request. The wearer of the first wearable device 200 may request the first wearable device 200 to make payment. For example, the wearer of the first wearable device 200 may request the first wearable device 200 to prepare for payment. The wearer of the first wearable device 200 may request that authority be granted such that the wearable device 200 makes payment by using user authentication.

In operation S1204, the first wearable device 200 according to an example may request the electronic device 100 to perform the authentication. The first wearable device 200 may request the electronic device 100 to perform personal authentication of the user and grant the same authentication to the first wearable device 200. For example, the first wearable device 200 may request the electronic device 100 to give authority to make payment.

In operation S1205, the electronic device 100 according to an example may check the authentication. The electronic device 100 may request the user to perform the user authentication. The electronic device 100 may request the user to input a password, a voice, or a pattern to prove that the user is a user of the electronic device 100. Alternatively, the electronic device 100 may receive biometric information such as iris information or fingerprint information of the user of the electronic device 100 or a person holding the electronic device 100 in his or her hand. The electronic device 100 may perform user personal authentication by confirming that the input biometric information is the same as that of the registered user.

In operation S1206, the electronic device 100 according to an example may respond to the authentication on the first wearable device 200. The electronic device 100 may emit an authentication signal for indicating that authentication is performed by using the communication unit 130. The first wearable device 200 may receive the authentication signal by using the communication unit 230.

In operation S1207, the first wearable device 200 according to an example may complete authentication. If the authentication signal is received, the first wearable device 200 may confirm that the wearer of the first wearable device 200 is the same person as the user of the electronic device 100. The state of the first wearable device 200 may be converted into an authenticated session state, which is the same state as the state in which personal authentication is completed. Accordingly, the user may make payment by using the first wearable device 200 without performing separate personal authentication on the first wearable device 200.

In operation S1208, the electronic device 100 according to an example may receive the PPG measurement request. The user may control the electronic device 100 such that the electronic device 100 measures the PPG signal. Alternatively, the first wearable device 200 may emit the PPG measurement request signal such that the electronic device 100 measures the PPG signal.

In operation S1209, the electronic device 100 according to an example may perform the PPG measurement task. The electronic device 100 may measure the PPG signal of the user of the electronic device 100 by using the sensor 110.

In operation S1210, the first wearable device 200 according to an example may perform the PPG measurement task. The first wearable device 200 may measure the PPG signal of the wearer of the first wearable device 200 by using the sensor 210.

In operation S1211, the electronic device 100 according to an example may transmit the PPG information to the first wearable device 200. The electronic device 100 may transmit data including the PPG signal of the user of the electronic device 100 to the first wearable device 200.

In operation S1212, the first wearable device 200 according to an example may analyze the PPG signal. The first wearable device 200 may compare the PPG signal measured by the electronic device 100 with the PPG signal measured by the first wearable device 200.

In an embodiment, the electronic device 100 may measure the PPG signal of the user according to an on-demand of the user of the electronic device 100. Further, the first wearable device 200 may measure the PPG signal of the user while the user is wearing the first wearable device. Accordingly, after initial personal authentication in the electronic device 100, the first wearable device 200 may maintain the authenticated session if the PPG measurement result remain the same.

In operation S1213, the first wearable device 200 according to an example may check whether it maintains the wearing state. If the PPG signal is measured, the first wearable device 200 may confirm that the wearer is continuously wearing the first wearable device 200. If getting out of the wearing state, the first wearable device 200 may release the authenticated session.

In operation S1214, the first wearable device 200 according to an example may maintain the authentication state while it maintains the wearing state. The first wearable device 200 may maintain the authenticated session by maintaining the authority granted by the electronic device 100 to the first wearable device while the wearer and the wearing state remain unchanged.

For example, the first wearable device 200 may be granted authority by the electronic device 100 by using a payment application (e.g., Samsung Pay) and may maintain a state in which payment is possible for a predetermined period of time (e.g., 30 minutes). After the predetermined time elapses, the granted authority may be terminated in the first wearable device 200 and thus the authenticated session may be released. Before the authenticated session of the first wearable device 200 is released, the electronic device 100 may receive a PPG measurement request from the user and may transmit the measurement result to the first wearable device 200. The first wearable device 200 may analyze the correlation between the PPG signal of the electronic device 100 and the PPG signal of the first wearable device 200. The first wearable device 200 may maintain the authenticated session if the correlation between the PPG signals is equal to or greater than a specified correlation, and thus, payment may be made without separate re-authentication even after a predetermined period of time if the first wearable device 200 maintains the wearing state by the wearer.

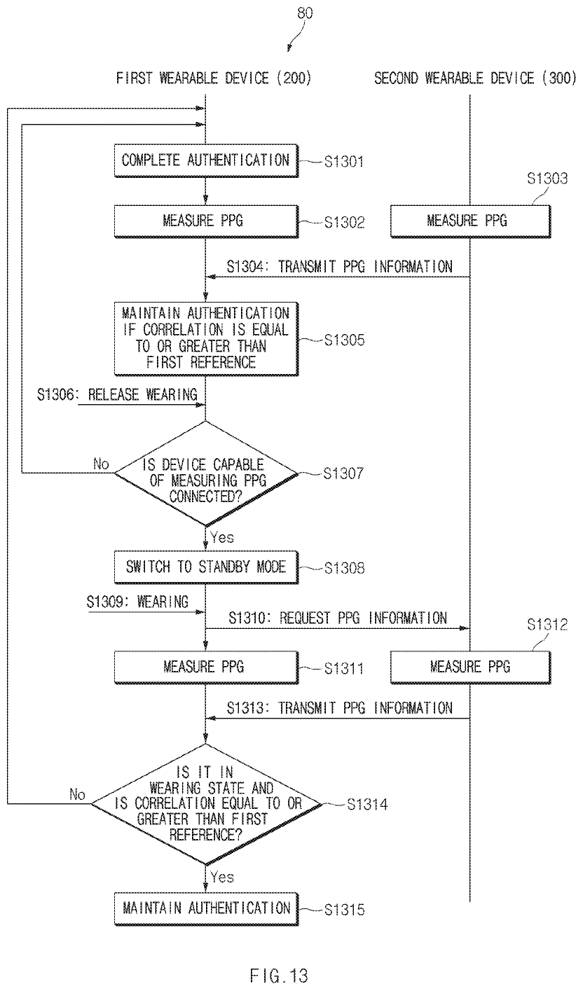

FIG. 13 is a flowchart 80 illustrating a process of authenticating the first wearable device 200 by a second wearable device 300 according to an embodiment.

In operation S1301, the first wearable device 200 according to an example may complete the authentication. The first wearable device 200 may be in a state in which authority is granted by the electronic device 100. Alternatively, the first wearable device 200 may complete the authentication by the wearer directly inputting a password, a pattern, or a PIN number.

In operation S1302, the first wearable device 200 according to an example may perform the PPG measurement task. The first wearable device 200 may measure the PPG signal of the wearer of the first wearable device 200 by using the sensor 210.

In operation S1303, the second wearable device 300 according to an example may perform the PPG measurement task. The second wearable device 300 may measure the PPG signal of the wearer of the second wearable device 300 by using a sensor.

In operation S1304, the second wearable device 300 according to an example may transmit the PPG information to the first wearable device 200.

In operation S1305, the first wearable device 200 according to an example may maintain the authentication if the correlation is equal to or greater than a first specified correlation. The first wearable device 200 may maintain an authenticated session if the correlation between the PPG signal measured by the first wearable device 200 and the PPG signal measured by the second wearable device 300 is equal to or greater than a specified correlation.

In operation S1306, the first wearable device 200 according to an example may be removed. The user may remove the first wearable device 200, which is in a state of maintaining the authenticated session. For example, if the first wearable device 200 is a watch, the user may remove (wrist-off) the wearable device 200 from the wrist.

In operation S1307, the first wearable device 200 according to an example may check whether it is connected with a device capable of measuring the PPG. If the first wearable device 200 is not connected with the device capable of measuring the PPG, the authenticated session may be released. For example, if the authenticated session of the first wearable device 200 is released, the wearer of the first wearable device 200 has to perform personal authentication again to make payment using the first wearable device 200.

In operation S1308, if the first wearable device 200 according to an example is connected with a device capable of measuring the PPG, the first wearable device 200 may be switched to a stand-by mode. The stand-by mode is a state in which the wearer of the first wearable device 200 removes the first wearable device 200 and thus may not use the granted authority, but the first wearable device 200 may be in a state of being capable of communication with a device capable of measuring the PPG. The first wearable device 200 may maintain a state in which returning to the authentication completion session later without completely ending the authenticated session in the stand-by mode.

In operation S1309, the first wearable device 200 according to an example may be worn. The user may wear the first wearable device 200, which is in a state of maintaining the stand-by mode again. For example, if the first wearable device 200 is a watch, the user may wear (wrist-on) the first wearable device 200 on the wrist again.

In operation S1310, the first wearable device 200 according to an example may request the second wearable device 300 to transmit the PPG information. The first wearable device 200 may instruct the second wearable device 300 to start measuring the PPG.

In operation S1311, the first wearable device 200 according to an example may perform the PPG measurement task. The first wearable device 200 may measure the PPG signal of the wearer of the first wearable device 200 by using the sensor 210.

In operation S1312, the second wearable device 300 according to an example may perform the PPG measurement task. The second wearable device 300 may measure the PPG signal of the wearer of the second wearable device 300 by using a sensor.

In operation S1313, the second wearable device 300 according to an example may transmit the PPG information to the first wearable device 200.

In operation S1314, the first wearable device 200 according to an example may check whether the wearable device 200 is in the wearing state and whether the correlation is equal to or greater than the first specified correlation. If the first wearable device 200 is not in the wearing state, the first wearable device 200 may release the authenticated session. In addition, if the correlation between the PPG signal measured by the first wearable device 200 and the PPG signal measured by the second wearable device 300 is less than the first specified correlation, the first wearable device 200 may release the authenticated session.

In operation S1315, the first wearable device 200 according to an example may maintain the authentication if the wearing state is maintained and the correlation is equal to or greater than the first specified correlation. The second wearable device 300 may continuously maintain the wearing state. For example, the second wearable device 300 may be the earbuds. If the correlation between the PPG signal measured by the first wearable device 200 and the PPG signal measured by the second wearable device 300 is greater than the first specified correlation, the first wearable device 200 may confirm that it is worn by the same person who wears the second wearable device 300.

In an embodiment, if the wearer is wearing a plurality of wearable devices 200 and 300 capable of measuring the PPG signal, the PPG signal may be measured all the time by using the wearable devices 200 and 300. One wearable device 200 may periodically receive the PPG signal measurement result from the other wearable device 300. If the correlation of the PPG signals is equal to or greater than the specified correlation, any one wearable device 200 may maintain the authenticated session of the wearable device 200.

In an embodiment, if one wearable device 200 is removed and the other wearable device 300 remains worn, the wearable device 200 that is removed may be switched to the stand-by mode. If the user re-wears the wearable device 200 that has been removed within a preset time, the re-worn wearable device 200 may calculate the correlation between the PPG signals measured by both wearable devices 200 and 300. If the correlation between the calculated PPG signals is equal to or greater than the specified correlation, the authenticated session of the re-worn wearable device 200 may be maintained.

For example, the PPG signal measured by the earbuds that are being worn may cause the watch to be switched to the stand-by mode when being removed. If the watch in the stand-by mode is worn again, the PPG signal measured by the watch and the PPG signal measured by the earbuds may be compared again. If the correlation between the PPG signal measured by the watch and the PPG signal measured by the earbuds is equal to or greater than the specified correlation, the watch may confirm that the wearer of the earbuds and the wearer of the watch are the same person. In this case, the authenticated session of the watch is maintained, and payment may be made by the watch even without a separate re-authenticated process.

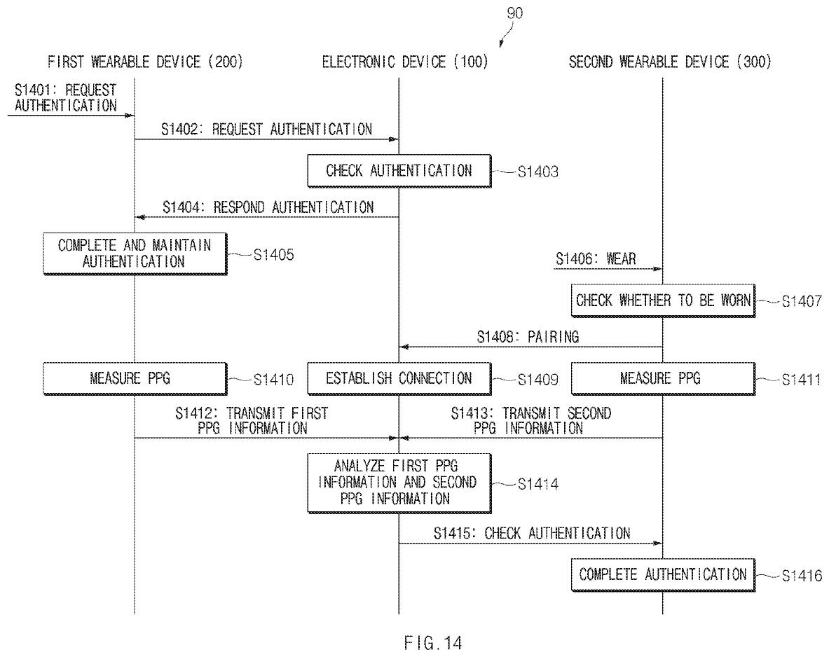

FIG. 14 is a flowchart 90 illustrating a process of authenticating the second wearable device 300 by the first wearable device 200 according to an embodiment.

In an embodiment, when the second wearable device 300 is connected in a state in which personal authentication is performed in the first wearable device 200, authentication may be selectively performed by using the electronic device 100 or the first wearable device 200 that is easily connectable according to the specified correlation of the PPG signal. In FIG. 14, a case of authenticating the second wearable device 300 by using the electronic device 100 is illustrated.

In operation S1401, the first wearable device 200 according to an example may receive an authentication request. The first wearable device 200 may receive the authentication request from the user.

In operation S1402, the first wearable device 200 according to an example may request the electronic device 100 to perform the authentication.

For example, if the electronic device 100 is a smartphone and the first wearable device 200 is a smart watch, the user may pair the smartphone with the smart watch using a biometric signal (e.g., PPG signal). The smartphone and the smart watch may transmit and receive data with each other by using a communication unit. The smart watch may request the smartphone to perform the user authentication. For example, the smart watch may request the smartphone to grant payment authority in order to make payment.

In operation S1403, the electronic device 100 according to an example may check whether to be authenticated. For example, if the electronic device 100 is a smartphone and the first wearable device 200 is a smart watch, the user may perform the authentication by inputting an authentication means (e.g., fingerprint, iris, password, pin, pattern) into the smartphone.

In operation S1404, the electronic device 100 according to an example may transmit, to the first wearable device 200, a response to the authentication request. The electronic device 100 may output a signal for authenticating that the wearer of the first wearable device 200 is the same person as the wearer of the electronic device 100.

For example, if the electronic device 100 is a smartphone and the first wearable device 200 is a smart watch, it may be proved that the smartphone and the smart watch are being used by the same user using the authentication means.

In operation S1405, the first wearable device 200 according to an example may maintain authentication after completing authentication. The first wearable device 200 may maintain an authenticated session if the wearer of the first wearable device 200 is authenticated as being the same person as the wearer of the electronic device 100.

For example, if it is proved that the same user is using the smartphone and the smart watch, the smartphone may update or merge the profile data of the user with the smart watch.

In operation S1406, the user may wear the second wearable device 300 according to an example. For example, if the second wearable device 300 is earbuds, the user may wear the earbuds in a state of carrying the smartphone and wearing the smart watch.

In operation S1407, the second wearable device 300 according to an example may check whether it is worn by the user. The second wearable device 300 may include a sensor capable of measuring a biometric signal (e.g., the PPG signal) of the user.

In operation S1408, the second wearable device 300 according to an example may be paired with the electronic device 100. For example, if the second wearable device 300 is the earbuds, the earbuds may be paired with the smartphone of the user by using the communication unit from a time when the earbuds detect the biometric signal of the user.

In operation S1409, the electronic device 100 according to an example may establish a connection. The electronic device 100 may be connected to the first wearable device 200 and the second wearable device 300 at the same time.

For example, from the standpoint of the smartphone, the smart watch and earbuds may be connected. In addition, if the user further wears a chest patch, the smartphone may be connected with the smart watch, earbuds, and chest patch at the same time.

In operation S1410, the first wearable device 200 according to an example may perform a PPG measurement task. The first wearable device 200 may measure the PPG signal of the wearer of the first wearable device 200 by using the sensor 210.

In operation S1411, the second wearable device 300 according to an example may perform the PPG measurement task. The second wearable device 300 may measure the PPG signal of the wearer of the second wearable device 300 by using a sensor.

In operation S1412, the first wearable device 200 according to an example may transmit the first PPG information to the electronic device 100. The first PPG information may be a PPG waveform measured by the first wearable device 200. The first PPG information may be a plurality of parameters generated by analyzing the PPG waveform measured by the first wearable device 200.

In operation S1413, the second wearable device 300 according to an example may transmit the second PPG information to the electronic device 100. The second PPG information may be a PPG waveform measured by the second wearable device 300. The second PPG information may be a plurality of parameters generated by analyzing the PPG waveform measured by the second wearable device 300.

In operation S1414, the electronic device 100 according to an example may analyze the first PPG information and the second PPG information. The electronic device 100 may calculate the correlation between the first PPG information and the second PPG information. The electronic device 100 may determine whether the first PPG information and the second PPG information are information measured by the same user.

For example, a smartphone may receive PPG information from the smart watch and the earbuds, respectively. The smartphone may determine whether the correlation between the PPG information received from the smart watch and the PPG information received from the earbuds is equal to or greater than the specified correlation.

In operation S1415, the electronic device 100 according to an example may check authentication of the second wearable device 300. If the first PPG information and the second PPG information are information measured by the same user, the electronic device 100 may determine that the first wearable device 200 and the second wearable device 300 are worn by the same user. If the first wearable device 200 and the second wearable device 300 are worn by the same user, the electronic device 100 may authenticate the second wearable device 300 or may grant the same authority as the wearable device 200 to the second wearable device 300.

For example, if the correlation between the PPG information received from the smart watch and the PPG information received from the earbuds is equal to or greater than the specified correlation, the smartphone may determine that the smart watch and the earbuds are worn by the same user. Even if the earbuds do not request separate authentication, the smartphone may grant the same authority as the smart watch to the earbuds if the same user wears them.

In operation S1416, the second wearable device 300 according to an example may complete authentication. The second wearable device 300 may be granted the same authority as the electronic device 100. The second wearable device 300 may update profile information with the electronic device 100.

In an embodiment, the connection between the electronic device 100 and the first wearable device 200 may be disconnected in a state in which the electronic device 100 and the second wearable device 300 are connected. Then, the electronic device 100 and the first wearable device 200 may be reconnected. The electronic device 100 may determine whether the wearer of the first wearable device 200 is the same person as the user of the electronic device 100 based on the PPG information provided from the second wearable device 300 maintaining the connection state. The electronic device 100 may determine the validity of the first wearable device 200 by using biometric information provided from the second wearable device 300 and may complete authentication of the first wearable device 200. The electronic device 100 according to an embodiment may authenticate the wearable device connected with the electronic device 100 based on the biometric information measured by at least one wearable device connected with the electronic device 100.

In an embodiment, in a state in which the first wearable device 200 and the second wearable device 300 are connected to the electronic device 100, the electronic device 100 may be connected with a third wearable device 400 capable of measuring biometric information (e.g., PPG information). The third wearable device 400 may complete authentication using biometric information received from at least one of the first wearable device 200 or the second wearable device 300. At least one wearable device connected with the electronic device according to an embodiment may authenticate the wearable device connected with the electronic device 100 based on the biometric information obtained by measurement.

The electronic device 100 and at least one wearable device connected to the electronic device 100 according to an example may simultaneously measure biometric information of the user and the wearable device connected with the electronic device 100. Accordingly, the user may perform user authentication on the wearable device connected with the electronic device 100 with high reliability. In addition, the electronic device 100 according to an example may grant authority to the wearable device connected with the electronic device 100 without a separate additional authentication means.

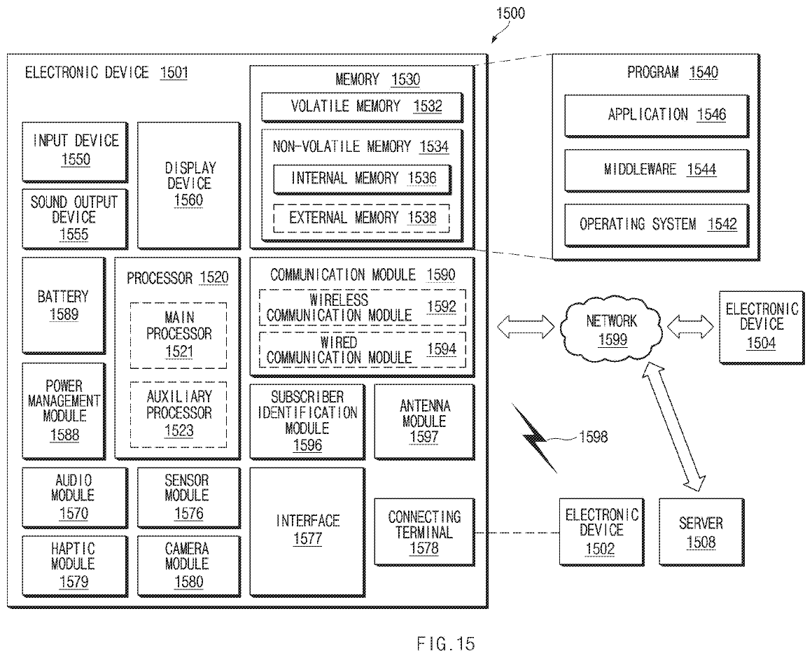

FIG. 15 is a block diagram illustrating an electronic device 1501 in a network environment 1500 according to various embodiments. Referring to FIG. 15, the electronic device 1501 in the network environment 1500 may communicate with an electronic device 1502 via a first network 1598 (e.g., a short-range wireless communication network), or an electronic device 1504 or a server 1508 via a second network 1599 (e.g., a long-range wireless communication network). According to an embodiment, the electronic device 1501 may communicate with the electronic device 1504 via the server 1508. According to an embodiment, the electronic device 1501 may include a processor 1520, memory 1530, an input device 1550, a sound output device 1555, a display device 1560, an audio module 1570, a sensor module 1576, an interface 1577, a haptic module 1579, a camera module 1580, a power management module 1588, a battery 1589, a communication module 1590, a subscriber identification module (SIM) 1596, or an antenna module 1597. In some embodiments, at least one (e.g., the display device 1560 or the camera module 1580) of the components may be omitted from the electronic device 1501, or one or more other components may be added in the electronic device 1501. In some embodiments, some of the components may be implemented as single integrated circuitry. For example, the sensor module 1576 (e.g., a fingerprint sensor, an iris sensor, or an illuminance sensor) may be implemented as embedded in the display device 1560 (e.g., a display).

The processor 1520 may execute, for example, software (e.g., a program 1540) to control at least one other component (e.g., a hardware or software component) of the electronic device 1501 coupled with the processor 1520, and may perform various data processing or computation. According to one embodiment, as at least part of the data processing or computation, the processor 1520 may load a command or data received from another component (e.g., the sensor module 1576 or the communication module 1590) in volatile memory 1532, process the command or the data stored in the volatile memory 1532, and store resulting data in non-volatile memory 1534. According to an embodiment, the processor 1520 may include a main processor 1521 (e.g., a central processing unit (CPU) or an application processor (AP)), and an auxiliary processor 1523 (e.g., a graphics processing unit (GPU), an image signal processor (ISP), a sensor hub processor, or a communication processor (CP)) that is operable independently from, or in conjunction with, the main processor 1521. Additionally or alternatively, the auxiliary processor 1523 may be adapted to consume less power than the main processor 1521, or to be specific to a specified function. The auxiliary processor 1523 may be implemented as separate from, or as part of the main processor 1521.

The auxiliary processor 1523 may control at least some of functions or states related to at least one component (e.g., the display device 1560, the sensor module 1576, or the communication module 1590) among the components of the electronic device 1501, instead of the main processor 1521 while the main processor 1521 is in an inactive (e.g., sleep) state, or together with the main processor 1521 while the main processor 1521 is in an active state (e.g., executing an application). According to an embodiment, the auxiliary processor 1523 (e.g., an image signal processor or a communication processor) may be implemented as part of another component (e.g., the camera module 1580 or the communication module 1590) functionally related to the auxiliary processor 1523.

The memory 1530 may store various data used by at least one component (e.g., the processor 1520 or the sensor module 1576) of the electronic device 1501. The various data may include, for example, software (e.g., the program 1540) and input data or output data for a command related thererto. The memory 1530 may include the volatile memory 1532 or the non-volatile memory 1534.

The program 1540 may be stored in the memory 1530 as software, and may include, for example, an operating system (OS) 1542, middleware 1544, or an application 1546.

The input device 1550 may receive a command or data to be used by other component (e.g., the processor 1520) of the electronic device 1501, from the outside (e.g., a user) of the electronic device 1501. The input device 1550 may include, for example, a microphone, a mouse, or a keyboard.

The sound output device 1555 may output sound signals to the outside of the electronic device 1501. The sound output device 1555 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 1560 may visually provide information to the outside (e.g., a user) of the electronic device 1501. The display device 1560 may include, for example, a display, a hologram device, or a projector and control circuitry to control a corresponding one of the display, hologram device, and projector. According to an embodiment, the display device 1560 may include touch circuitry adapted to detect a touch, or sensor circuitry (e.g., a pressure sensor) adapted to measure the intensity of force incurred by the touch.

The audio module 1570 may convert a sound into an electrical signal and vice versa. According to an embodiment, the audio module 1570 may obtain the sound via the input device 1550, or output the sound via the sound output device 1555 or a headphone of an external electronic device (e.g., an electronic device 1502) directly (e.g., wiredly) or wirelessly coupled with the electronic device 1501.

The sensor module 1576 may detect an operational state (e.g., power or temperature) of the electronic device 1501 or an environmental state (e.g., a state of a user) external to the electronic device 1501, and then generate an electrical signal or data value corresponding to the detected state. According to an embodiment, the sensor module 1576 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1577 may support one or more specified protocols to be used for the electronic device 1501 to be coupled with the external electronic device (e.g., the electronic device 1502) directly (e.g., wiredly) or wirelessly. According to an embodiment, the interface 1577 may include, for example, a high definition multimedia interface (HDMI), a universal serial bus (USB) interface, a secure digital (SD) card interface, or an audio interface.

A connecting terminal 1578 may include a connector via which the electronic device 1501 may be physically connected with the external electronic device (e.g., the electronic device 1502). According to an embodiment, the connecting terminal 1578 may include, for example, a HDMI connector, a USB connector, a SD card connector, or an audio connector (e.g., a headphone connector).

The haptic module 1579 may convert an electrical signal into a mechanical stimulus (e.g., a vibration or a movement) or electrical stimulus which may be recognized by a user via his tactile sensation or kinesthetic sensation. According to an embodiment, the haptic module 1579 may include, for example, a motor, a piezoelectric element, or an electric stimulator.

The camera module 1580 may capture a still image or moving images. According to an embodiment, the camera module 1580 may include one or more lenses, image sensors, image signal processors, or flashes.

The power management module 1588 may manage power supplied to the electronic device 1501. According to one embodiment, the power management module 1588 may be implemented as at least part of, for example, a power management integrated circuit (PMIC).

The battery 1589 may supply power to at least one component of the electronic device 1501. According to an embodiment, the battery 1589 may include, for example, a primary cell which is not rechargeable, a secondary cell which is rechargeable, or a fuel cell.

The communication module 1590 may support establishing a direct (e.g., wired) communication channel or a wireless communication channel between the electronic device 1501 and the external electronic device (e.g., the electronic device 1502, the electronic device 1504, or the server 1508) and performing communication via the established communication channel. The communication module 1590 may include one or more communication processors that are operable independently from the processor 1520 (e.g., the application processor (AP)) and supports a direct (e.g., wired) communication or a wireless communication. According to an embodiment, the communication module 1590 may include a wireless communication module 1592 (e.g., a cellular communication module, a short-range wireless communication module, or a global navigation satellite system (GNSS) communication module) or a wired communication module 1594 (e.g., a local area network (LAN) communication module or a power line communication (PLC) module). A corresponding one of these communication modules may communicate with the external electronic device via the first network 1598 (e.g., a short-range communication network, such as Bluetooth™, wireless-fidelity (Wi-Fi) direct, or infrared data association (IrDA)) or the second network 1599 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 1592 may identify and authenticate the electronic device 1501 in a communication network, such as the first network 1598 or the second network 1599, using subscriber information (e.g., international mobile subscriber identity (IMSI)) stored in the subscriber identification module 1596.

The antenna module 1597 may transmit or receive a signal or power to or from the outside (e.g., the external electronic device) of the electronic device 1501. According to an embodiment, the antenna module 1597 may include one or more antennas, and, therefrom, at least one antenna appropriate for a communication scheme used in the communication network, such as the first network 1598 or the second network 1599, may be selected, for example, by the communication module 1590 (e.g., the wireless communication module 1592). The signal or the power may then be transmitted or received between the communication module 1590 and the external electronic device via the selected at least one antenna.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 1501 and the external electronic device 1504 via the server 1508 coupled with the second network 1599. Each of the electronic devices 1502 and 1504 may be a device of a same type as, or a different type, from the electronic device 1501. According to an embodiment, all or some of operations to be executed at the electronic device 1501 may be executed at one or more of the external electronic devices 1502, 1504, or 1508. For example, if the electronic device 1501 should perform a function or a service automatically, or in response to a request from a user or another device, the electronic device 1501, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 1501. The electronic device 1501 may provide the outcome, with or without further processing of the outcome, as at least part of a reply to the request. To that end, a cloud computing, distributed computing, or client-server computing technology may be used, for example.

The electronic device according to various embodiments may be one of various types of electronic devices. The electronic devices may include, for example, a portable communication device (e.g., a smart phone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 1540) including one or more instructions that are stored in a storage medium (e.g., internal memory 1536 or external memory 1538) that is readable by a machine (e.g., the electronic device 1501). For example, a processor (e.g., the processor 1520) of the machine (e.g., the electronic device 1501) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

1. An electronic device comprising:
a sensor which measures first biometric information;
a communication unit which receives second biometric information measured by at least one wearable device; and
a processor operatively connected with the sensor and the communication unit, wherein the processor calculates a correlation between the first biometric information and the second biometric information, grant, to at least one wearable device, an authenticated authority based on the correlation, the authenticated authority being an authority to approve performance of a specified operation, and sets an authenticated authority level that is a step classified based on reliability of the authenticated authority.

2. The electronic device of claim 1, wherein the processor grants, to the at least one wearable device, a first authenticated authority level if the correlation is equal to or greater than a specified correlation, the first authenticated authority level being an authenticated authority level having the same reliability as the electronic device for which user authentication has been completed.

3. The electronic device of claim 1, wherein the processor determines that a user of the electronic device and a wearer of the at least one wearable device are the same person if the correlation is equal to or greater than a specified correlation.

4. The electronic device of claim 1, wherein the processor:
calculates first photoplethysmogram (PPG) information based on the first biometric information and second PPG information based on the second biometric information; and
grants a first authenticated authority level to the at least one wearable device if a correlation between the first PPG information and the second PPG information is equal to or greater than a specified correlation.

5. The electronic device of claim 1, wherein the processor:
calculates first PPG information based on the first biometric information and second PPG information based on the second biometric information; and
grants a second authenticated authority level having a lower or more limited reliability than a first authenticated authority level to the at least one wearable device if a correlation between the first PPG information and the second PPG information is less than a specified correlation.

6. The electronic device of claim 1, wherein the first biometric information and the second biometric information are information including at least one of PPG information, heart rate information, or electrocardiogram (ECG) information.

7. The electronic device of claim 1, wherein the processor calculates an amplitude and a phase of the first biometric information and an amplitude and a phase of the second biometric information.

8. The electronic device of claim 1, wherein information on at least one of amplitudes of waveforms, intervals of waveforms, or change rates of waveforms of the first biometric information and the second biometric information is extracted by applying a derivative to at least a portion of the first biometric information and the second biometric information.

9. The electronic device of claim 1, wherein the first biometric information and the second biometric information are represented by a plurality of frequency components, and a plurality of parameters are calculated using a plurality of peaks having a frequency of an integer multiple of a specified frequency among the plurality of frequency components.

10. The electronic device of claim 1, wherein the processor:
calculates a degree of matching level between a heart rate trend measured by the electronic device and a heart rate trend measured by the at least one wearable device; and
determines whether a user of the electronic device and a wearer of the at least one wearable device are the same person using the degree of matching.

11. The electronic device of claim 1, wherein the processor:
grants a first authenticated authority level to the at least one wearable device if a change rate of a heart rate measured by the electronic device and a change rate of a heart rate measured by the at least one wearable device match during a preset duration time or more; and
if the change rate of the heart rate measured by the electronic device and the change rate of the heart rate measured by the at least one wearable device are equal to or greater than a preset change amount.

12. A method for controlling an electronic device, comprising:
pairing with at least one wearable device;
measuring first biometric information by the electronic device;
requesting the at least one wearable device to measure biometric information;
measuring second biometric information by the at least one wearable device;
receiving the second biometric information from the at least one wearable device and comparing the first biometric information with the second biometric information; and
calculating a correlation between the first biometric information and the second biometric information, granting, to at least one wearable device, an authenticated authority that is an authority to approve performance of a specified operation, based on the correlation, and setting an authenticated authority level that is a step classified based on reliability of the authenticated authority.

13. The method for controlling an electronic device of claim 12, further comprising:
granting, to the at least one wearable device, a first authenticated authority level that is an authenticated authority level having the same reliability as the electronic device for which user authentication has been completed, if it is determined that a user of the electronic device and a wearer of the at least one wearable device are the same person.

14. The method for controlling an electronic device of claim 12, further comprising:
requesting the at least one wearable device to perform additional authentication if a second authenticated authority level having a lower or more limited reliability than a first authenticated authority level is granted to the at least one wearable device.

15. The method for controlling an electronic device of claim 12, further comprising:
maintaining an authenticated session of the at least one wearable device while a user is wearing the at least one wearable device, after the granting of the authenticated authority to the at least one wearable device has been completed.
